(19) United States
(12) Patent Application Publication
(10) Pub. No.: US 2026/0137908 A1
(43) Pub. Date: May 21, 2026

(54) CATHETER ASSEMBLY WITH MOVABLE DISTAL TIP SUPPORT

(71) Applicant: Becton, Dickinson and Company, Franklin Lakes, NJ (US)

(72) Inventors: Jonathan Karl Burkholz, Salt Lake City, UT (US); Weston F. Harding, Lehi, UT (US)

(73) Assignee: Becton, Dickinson and Company, Franklin Lakes, NJ (US)

(21) Appl. No.: 18/949,274

(22) Filed: Nov. 15, 2024

(51) Int. Cl. A61M 25/06; A61M 25/00

(52) U.S. Cl. CPC A61M 25/0606; A61M 25/0023; A61M 25/0043; A61M 25/0067; A61M 2025/0059; A61M 2205/0233

(57) ABSTRACT

Catheter assemblies vascular access devices using such assemblies are disclosed. The catheter assembly comprises a catheter distal tip support element movably disposed within a catheter lumen and over the introducer needle. The catheter distal tip support element is movable with respect to the catheter and the introducer needle and having a support stepped surface defining a support distal surface configured to contact a shoulder of the catheter and to advance the catheter in distal direction and prevent kinking or buckling of the second material of the catheter as the catheter is advanced into the vasculature of the patient.

### TECHNICAL FIELD

The present disclosure pertains to catheter assemblies including a relatively soft catheter positioned over an introducer needle and a catheter distal tip support that is relatively hard and movable with respect to the catheter positioned over the introducer needle and an advancement assembly configured to advance the catheter to a hooded position.

### BACKGROUND

Intravenous (IV) catheters are commonly used for a variety of infusion therapies. For example, IV catheters is used for infusing normal saline solution, various medicaments, or total parenteral nutrition into a patient. IV catheters may also be used for withdrawing blood from the patient.

A common type of IV catheter is an over-the-needle peripheral IV catheter. As its name implies, over-the-needle peripheral IV catheters comprise catheter tubing mounted tightly over an introducer needle having a sharp distal tip. The introducer needle and the IV catheter are generally inserted at a shallow angle through the skin into vasculature of the patient with a bevel of the introducer needle facing away from the skin of the patient. During insertion, the sharp distal tip of the introducer needle is distal to a distal end of the IV catheter. Thus, insertion of the IV catheter into the vasculature may follow piercing of the vasculature by the introducer needle.

Generally, the introducer needle is constructed of metal, while the catheter tubing is constructed of a softer material, for example, a thermoplastic or elastomeric material. Softer catheter tubing materials, as opposed to harder materials, are more comfortable for the patient, cause less irritation, and improve indwelling. However, if the catheter tubing is too soft, its columnar strength may not be great enough to resist forces required to penetrate the skin and tissue of the patient.

In some instances, when the introducer needle and the IV catheter are inserted into the patient, there is a tendency for the catheter tubing to buckle, peel back, or move proximally away from the distal tip of the introducer needle. This may occur due to the skin or tissue of the patient pushing on a distal tip of the IV catheter during the insertion process. When the catheter tubing peels back or moves proximally away from the distal tip of the introducer needle, the catheter tubing may adopt an enlarged cross-section, which may require greater insertion force, increased dilation at the insertion site, and/or increased pain for the patient.

There is a need to provide catheter assemblies that improve insertion success, reduce placement related complications, and provide a softer, longer dwelling catheter tubing than those provided with traditionally placed short and mid-line peripheral IV catheters.

### SUMMARY

One aspect of the disclosure relates to a catheter assembly configured for insertion into vasculature of a patient. In one embodiment, the catheter assembly comprises a catheter, comprising a catheter stepped surface having a distal surface with a first inner diameter, a proximal surface with a second inner diameter greater than the first inner diameter, and a shoulder disposed between the distal surface and the proximal surface. The catheter further comprises an elongate tubular portion proximate the catheter stepped surface and having the second inner diameter of the proximal surface, wherein the shoulder, the proximal surface, and the distal surface are constructed of a first material having a Shore durometer that is equal to or greater than a Shore durometer of a second material of the elongate tubular portion, the catheter having a catheter lumen extending therethrough, wherein the catheter stepped surface is disposed within the catheter lumen.

The catheter assembly further comprises an introducer needle disposed within the catheter lumen, the introducer needle comprising a sidewall defining a proximal end, a distal tip, and a lumen extending through from the proximal end to the distal tip. The catheter assembly further comprises a catheter distal tip support element movably disposed within the catheter lumen and over the introducer needle, the catheter distal tip support element comprising a support material having a higher Shore durometer than the Shore durometer of the second material of the elongate tubular portion of the catheter, the catheter distal tip support element movable with respect to the catheter and the introducer needle and having a support stepped surface defining a support distal surface configured to contact the shoulder of the catheter and to advance the catheter in distal direction and prevent kinking or buckling of the second material of the catheter as the catheter is advanced into the vasculature of the patient.

### BRIEF DESCRIPTION OF THE DRAWINGS

FIG. 1A is a top view of a catheter assembly according to one or more embodiments;

FIG. 1B is a cross-sectional view of a catheter of the catheter assembly of FIG. 1A, according to one or more embodiments;

FIG. 1C is an upper perspective view of the distal end of the catheter of FIG. 1B, according to one or more embodiments;

FIG. 1D is an upper perspective view of a distal end of an example introducer needle of the catheter assembly of FIG. 1A, according to one or more embodiments;

FIG. 2 is a cross-sectional view of a portion of a catheter assembly including an introducer needle, a catheter and a reinforced distal catheter tip according to one or more embodiments;

FIG. 3A is a cross-sectional view of the introducer needle and the catheter in FIG. 2 in a position prior into insertion to a patient;

FIG. 3B is a cross-sectional view of the introducer needle and the catheter shown in FIG. 2 with the catheter in a hooded position over the distal tip of the introducer needle;

FIG. 3C is a cross-sectional view of the introducer needle and the catheter shown in FIG. 2 with the catheter in a partially advanced position over the distal tip of the introducer needle;

FIG. 3D is a cross-sectional view of the introducer needle and the catheter shown in FIG. 2 after the catheter has been fully advanced into the vasculature of a patient and the introducer needle has been removed from the catheter;

FIG. 4A is a cross-sectional view of the introducer needle and the catheter shown in FIG. 2 in a position prior to insertion into a patient;

FIG. 4B is a cross-sectional view of the introducer needle and the catheter shown in FIG. 2 with the catheter in a hooded position over the distal tip of the introducer needle;

FIG. 4C is a cross-sectional view of the introducer needle and the catheter shown in FIG. 2 with the catheter in a partially advanced position over the distal tip of the introducer needle;

FIG. 4 D is a cross-sectional view of the introducer needle and the catheter shown in FIG. 2 after the catheter has been placed in the vasculature of a patient and the introducer needle has been removed from the catheter;

FIG. 5A is a cross-sectional view of a second embodiment of a catheter assembly with a longer reinforced catheter distal tip support in an initial position;

FIG. 5B is a cross-sectional view of the catheter assembly in FIG. 5A showing the longer reinforced catheter tip;

FIG. 6A is a cross-sectional view of a third embodiment of a catheter assembly with a coil element providing catheter distal tip support;

FIG. 6B is a side view of the introducer needle shown catheter assembly in FIG. 6A showing the coil element that provides catheter distal tip support;

FIG. 6C is a cross-sectional view showing a variant of the catheter assembly shown in FIG. 6A;

FIG. 7A is a cross-sectional view of a fourth embodiment showing a catheter adapter assembly with a catheter distal tip support in an initial position;

FIG. 7B is a cross-sectional view of the catheter adapter assembly in FIG. 7A with the catheter in the hooded position;

FIG. 7C is a cross-sectional view of the catheter adapter assembly in FIG. 7A showing the catheter further advanced from the introducer needle;

FIG. 7D is a cross-sectional view of the catheter adapter assembly in FIG. 7A showing the introducer needle nearly fully withdrawn from the catheter after the catheter has been placed in the vasculature of a patient;

FIG. 7E is a cross-sectional view of the catheter adapter assembly in FIG. 7A showing the introducer needle fully withdrawn from the catheter after the catheter has been placed in the vasculature of a patient;

FIG. 8A is a cross-sectional view of a fifth embodiment showing a catheter adapter assembly with a catheter distal tip support in the form of an advancement sleeve instead of an advancement member that passes through the introducer needle to attach to the distal tip support sleeve via a notch or slot in the needle cannula in an initial position;

FIG. 8B is a cross-sectional view of the catheter adapter assembly in FIG. 8A with the catheter in the hooded position;

FIG. 9A is cross-sectional view of a sixth embodiment of a catheter assembly with a catheter distal tip support and a supplemental catheter distal tip support in an initial position; and

FIG. 9B is a cross-sectional view of the catheter assembly in FIG. 9A with the catheter in the hooded position.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Embodiments of the present disclosure provide catheter assemblies which can be used with a variety of vascular access devices (VAD) and in various methods and systems. One or more embodiments provide catheter assemblies that provide one or more of improved insertion success, reduced placement related complications, and provide a softer, longer dwelling a softer, longer dwelling catheter tubing than those provided with traditionally placed short and mid-line peripheral IV catheters. Embodiments of the catheter assembly and systems of this disclosure provide for placement of a VAD with a much softer, and longer dwelling, catheter material via an over-the-needle catheter placement device. This is accomplished by providing a movable distal catheter tip support and “hooding” feature and a softer, more compliant and kink resistant catheter material. One or more embodiments also provide a catheter assembly that is configured to place a longer catheter for deeper vein placements that often have a steeper insertion angle and tighter angle changes that can be susceptible to kinking or buckling. According to one or more embodiments, a catheter assembly and system is provided that is less complicated than existing catheter assemblies.

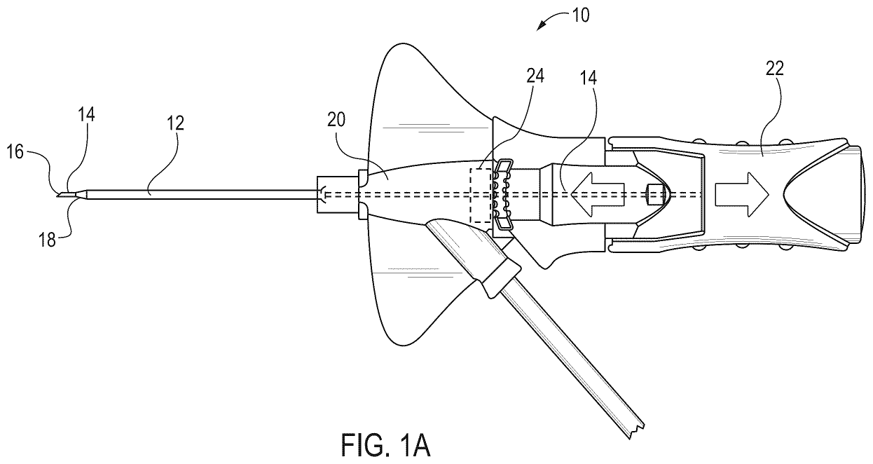

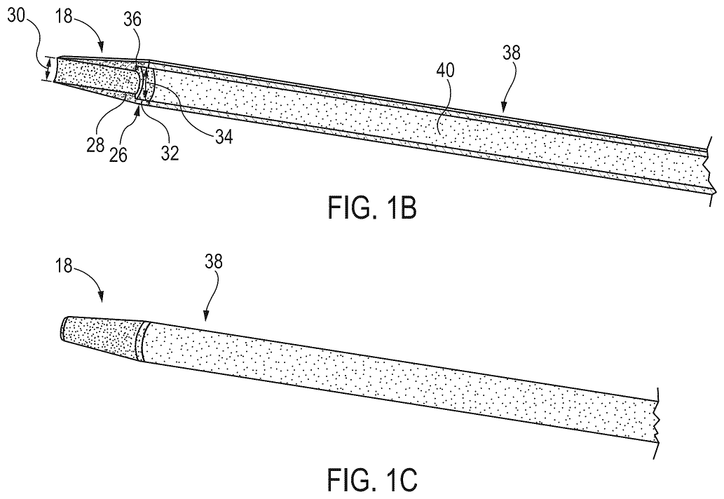

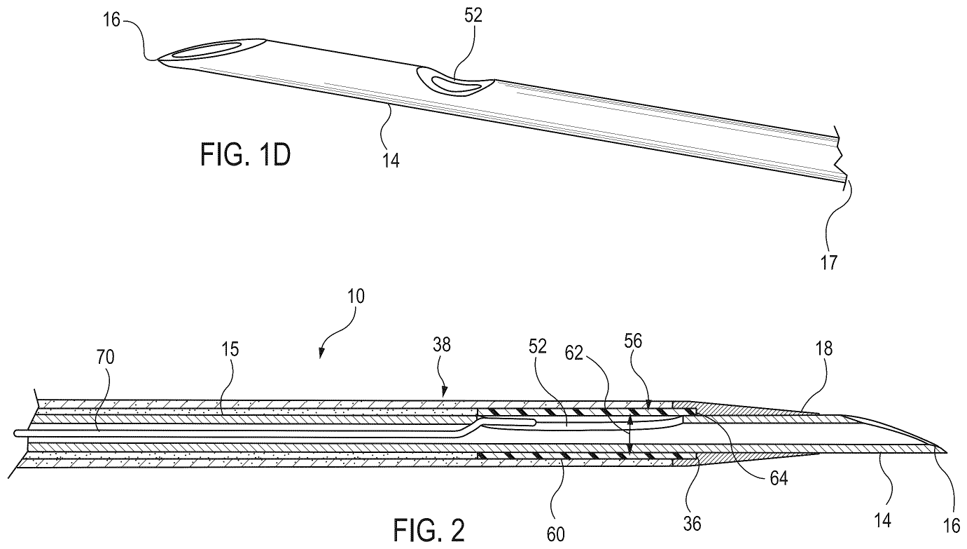

Embodiments of the present disclosure generally relate to a catheter assembly configured for insertion into vasculature of a patient and related devices and methods. Referring now to FIG. 1A, in one or more embodiments, the catheter assembly 10 includes catheter 12 positioned over an introducer needle 14. The catheter 12 may thus be referred to as an over-the-needle catheter. FIG. 1A illustrates the introducer needle 14 in an insertion position for insertion into a patient. The introducer needle 14 comprises a distal tip, a proximal end and a notch (or slot) 52. In one or more embodiments, when the introducer needle 14 is in the insertion position, a sharp distal tip 16 of the introducer needle 14 is disposed beyond a distal tip 18 of the over-the-needle catheter 12.

In one or more embodiments, the catheter assembly 10 includes a catheter adapter 20. As shown, a proximal end of the over-the-needle catheter 12 is secured within the catheter adapter 20, and the over-the-needle catheter 12 extends distally from the catheter adapter 20. A proximal end of the introducer needle 14 is secured within a needle hub 22 of the catheter assembly 10.

The catheter assembly 10 shown includes any type of catheter assembly having an over-the-needle catheter and introducer needle. Although the catheter assembly 10 illustrated in FIG. 1A is shown as having an integrated extension tube, it is understood that the catheter assembly 10 is straight or non-integrated. Non-limiting examples of integrated IV catheter systems includes, for example, the BD NEXIVA™ Closed IV Catheter System, the BD NEXIVA™ DIFFUSICS™ Closed IV Catheter System, or the Becton Dickinson PEGASUS™ Safety Closed IV Catheter System. A non-limiting example of a straight or non-integrated catheter assembly is the BD INSYTE™ AUTOGUARD™ Shielded IV Catheter. In one or more embodiments, the catheter assembly 10 includes any variety of needle safety mechanism, which may shield the distal tip 16 of the introducer needle 14.

In one or more embodiments, the catheter assembly 10 includes any suitable type of blood control septum, which is disposed within a lumen of the catheter adapter 20. FIG. 1A illustrates an example blood control septum 24, according to one or more embodiments. In one or more embodiments, the introducer needle 14 is proximally withdrawn through the septum 24 after the over-the-needle catheter 12 is properly placed within the vasculature. In one or more embodiments, the septum 24 is elastomeric. In one or more embodiments, the septum 24 may divide the lumen of the catheter adapter 20 into a proximal chamber and a distal chamber.

Referring now to FIG. 1B, in one or more embodiments, the over-the-needle catheter 12 includes the distal tip 18, which is constructed of the first material having a first Shore durometer value. In one or more embodiments, the first material is rigid or semi-rigid. In one or more embodiments, the over-the-needle catheter 12 includes a first stepped surface 26, which in some embodiments is referred to a catheter stepped surface 26 that is annular. In other embodiments, the stepped surface 26 may comprise one or more discrete protrusions protruding from an interior surface of the catheter lumen 40 of the over-the-needle catheter 12 that provide a stepped surface 26 shoulder. In one or more embodiments, the first stepped surface 26 includes a rigid or semi-rigid distal surface 28 with a first inner diameter 30, a proximal surface 32 with a second inner diameter 34 greater than the first inner diameter 30, and a rigid or semi-rigid first transition surface defining a shoulder 36 disposed between the distal surface 28 and the proximal surface 32. The first inner diameter 30 is uniform according to one or more embodiments. In some embodiments the second inner diameter 34 is uniform.

In one or more embodiments, an entirety of the distal tip is constructed of the first material, which may also be referred to as the distal tip material. In one or more embodiments, at least the distal surface 28 and the first transition surface defining the shoulder 36 of the first stepped surface 26 is part of the distal tip 18 of the over-the-needle catheter 12 and thus constructed of the first material. In one or more embodiments, the distal surface 28, the first transition surface defining the shoulder 36, and the proximal surface 32 of the first stepped surface 26 is part of the distal tip 18 and thus constructed of the first material. In some embodiments, the distal type includes fenestrations or openings (not shown).

In one or more embodiments, the over-the-needle catheter 12 includes an elongate tubular portion 38 proximate and coupled to the distal tip 18 of the over-the-needle catheter 12. In one or more embodiments, the tubular portion 38 is constructed of a second material. In one or more embodiments, a Shore durometer value of the first material is equal to or higher (a harder material) than a Shore durometer value of the second material. In one or more embodiments, the Shore durometer of the first material and the second material are equal, and in some embodiments, the first material and the second material comprise, consist of or consist essentially of the same material. In one or more embodiments, the distal tip 18 and the tubular portion 38 includes a catheter lumen 40 extending there through. In one or more embodiments, the first stepped surface 26 is disposed within the catheter lumen 40.

Referring now to FIG. 1C, in one or more embodiments, at least a portion of an outer surface of the distal tip 18 is tapered, which may facilitate entry of the over-the-needle catheter 12 into the skin and tissues of the patient behind the distal tip 16 of the introducer needle 14 as the distal tip 16 cuts a pathway.

Referring to FIG. 1D, the catheter assembly 10 includes the introducer needle 14, which in some embodiments includes the notch (or slot) 52 providing access to a needle lumen 54. In some embodiments, a portion of the introducer needle 14 proximate the notch (or slot) 52 may be spaced apart from an inner surface of the over-the-needle catheter 12 or tubular portion 38 to form a flashback pathway 56. In some embodiments, the flashback pathway 56 may be disposed within the catheter lumen 40 between an outer surface of the introducer needle 14 and an inner surface of the over-the-needle catheter 12. The introducer needle has a length extending from the distal tip.

In one or more embodiments, the first material and/or the second material includes a composite material. In one or more embodiments, the first material includes one or more of the following: silicone, polyurethane, metal, plastic, and another suitable material. In one or more embodiments, the second material may one or more of the following: silicone, polyurethane, metal, plastic, and another suitable material. In one or more embodiments, the first material includes a first polyurethane, and the second material includes a second polyurethane. In one or more embodiments, the first material includes a first silicone, and the second material includes a second silicone. In one or more embodiments, the first material includes metal and/or the second material includes plastic. In one or more embodiments, the second material includes a flexible material. In one more embodiments, the second material comprises a soft polymeric material, for example, silicone rubber, nylon, polyurethane, fluoroethylene polymer (FEP), polytetrafluoroethyene polymer (PTFE), polyethylene terephthalate (PET), latex, and thermoplastic elastomers.

In one or more embodiments, the second material includes a softer material than the first material, which is more comfortable for the patient and result in fewer indwelling problems. In one or more embodiments, the softer second material may allow the over-the-needle catheter 12 to be inserted into the vasculature longer before complications arise.

Referring to FIG. 2, the catheter assembly includes a catheter distal tip support element 60 movably disposed within the catheter lumen 40 of the over-the-needle catheter 12 and over the introducer needle 14. The catheter distal tip support element 60 comprises a support material having a Shore durometer that is equal to or greater than a Shore durometer of the second material which makes up the elongate tubular portion 38 of the over-the-needle catheter 12. In embodiments in which the Shore durometer of the first material is greater than the Shore durometer of the second material, and the first material is harder than the second material.

The catheter distal tip support element 60 is movable in a distal and proximal direction with respect to the over-the-needle catheter 12 and the introducer needle 14. The catheter distal tip support element 60 in some embodiments has an outer diameter 62 and a support distal surface 64 configured to contact the shoulder 36 of the over-the-needle catheter 12 and to advance the over-the-needle catheter 12 in distal direction and prevent kinking or buckling of the second material of the over-the-needle catheter 12 as the over-the-needle catheter 12 is advanced into the vasculature of the patient. In one or more embodiments, the support distal surface 64 may contact the shoulder 36 of the over-the-needle catheter 12 when the introducer needle 14 is in the insertion position, such that proximal movement of the distal tip 16 relative to the introducer needle 14 is inhibited and compressive forces necessary for the distal tip 16 to penetrate the skin are transferred to the distal tip 18 of the over-the-needle catheter 12. In one or more embodiments, the contact between the support distal surface 64 and the shoulder 36 when the introducer needle 14 is in the insertion position may allow the second material to be even softer than a standard catheter body. In one or more embodiments, the second material may allow the tubular portion 38 to bend when the catheter adapter 20 is secured to the skin of the patient, while the first material may facilitate successful insertion of the over-the-needle catheter 12 into the vasculature.

In one or more embodiments, the first transition surface defines a shoulder 36 that is disposed at about 90 degrees with respect to a longitudinal axis of the catheter assembly 10, as illustrated, for example, in FIG. 1B. In one or more embodiments, the first transition surface defining the shoulder 36 is disposed at an angle other than 90 degrees with respect to the longitudinal axis of the catheter assembly 10. In one or more embodiments, the first transition surface defining the shoulder 36 is smooth and/or planar. In one or more embodiments, the first transition surface defining the shoulder 36 is uneven, rough, or irregular. In one or more embodiments, the first transition surface defining the shoulder 36 is curved.

FIG. 2 is a cross-sectional view of a catheter assembly 10 including the introducer needle 14 and the over-the-needle catheter 12 with the distal tip 18 according to one or more embodiments.

The catheter assembly 10 is configured for insertion into vasculature of a patient. The catheter assembly 10 includes an over-the-needle catheter 12 which comprises the catheter stepped surface 26 having a distal surface 28 with a first inner diameter 30, a proximal surface 32 with a second inner diameter 34 greater than the first inner diameter 30, and a shoulder 36 disposed between the distal surface 28 and the proximal surface 32.

The catheter assembly 10 further comprises an elongate tubular portion 38 proximate the catheter stepped surface 26 and having the second inner diameter 34 of the proximal surface 32, wherein the shoulder 36, the proximal surface 32, and the distal surface 28 are constructed of a first material having a Shore durometer that is equal to or greater than a Shore durometer a second material of the elongate tubular portion 38. The over-the-needle catheter 12 has a catheter lumen 40 extending therethrough, and the catheter stepped surface 26 is disposed within the catheter lumen 40.

The catheter assembly 10 further comprises an introducer needle 14 disposed within the catheter lumen 40, the introducer needle 14 comprising a sidewall 15 defining a proximal end 17, a distal tip 16 and a needle lumen 54 extending through the sidewall 15 from the proximal end 17 to the distal tip 16.

The catheter assembly 10 further comprises a catheter distal tip support element 60 movably disposed within the catheter lumen 40 and over the introducer needle 14, the catheter distal tip support element 60 comprising a support material having a Shore durometer that is equal to or greater than a Shore durometer of the second material of the elongate tubular portion 38 of the catheter, the catheter distal tip support element 60 element movable with respect to the over-the-needle catheter 12 and the introducer needle 14 and having a support distal surface 64 configured to contact the shoulder 36 of the over-the-needle catheter 12 and to advance the over-the-needle catheter 12 in distal direction and prevent kinking or buckling of the second material of the catheter as the catheter is advanced into the vasculature of the patient,

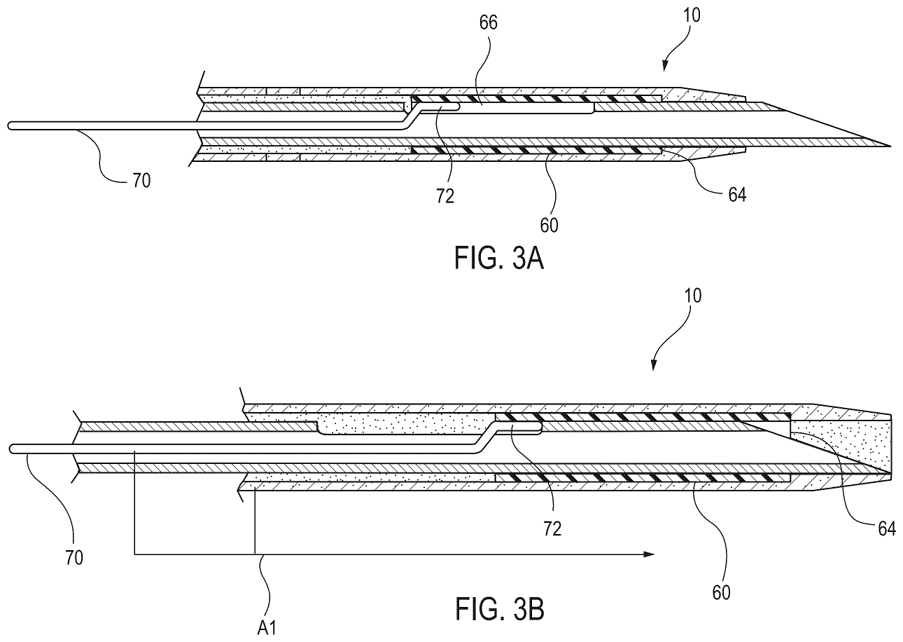

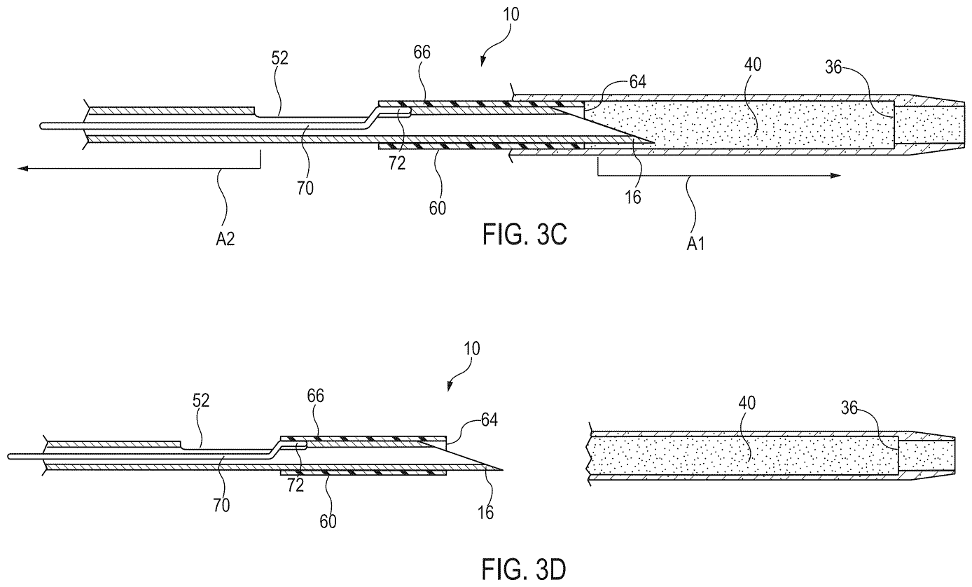

FIG. 3A is a cross-sectional view of the introducer needle 14 and the over-the-needle catheter 12 in FIG. 2 in a position prior into insertion to a patient, and FIG. 3B is a cross-sectional view of the introducer needle 14 and the over-the-needle catheter 12 shown in FIG. 2 with the over-the-needle catheter 12 in a hooded position over the distal tip of the introducer needle 14. Advantageously, according to one or more embodiments, with the shoulder 36 and the support distal surface 64 in a mechanically pre-defined position, the catheter assembly 10 is configured to provide proper and consistent hooding to advance the catheter in by advancing the advancement member 70 in the direction of arrow A1 while providing distal tip 18 of the over-the-needle catheter 12, especially the softer elongate tubular portion 38, support to be able to successfully advance the over-the-needle catheter 12 comprising a much softer material than the distal tip 18 of the over-the-needle catheter 12. Thus, embodiments provide a catheter assembly 10 comprising a relatively soft catheter 12 configured for placement over an introducer needle 14 and a catheter distal tip support that is relatively hard and movable with respect to the catheter. The movable catheter distal tip support provides a mechanically defined position configured to provide proper and consistent hooding while providing catheter distal tip support to be able to successfully advance a catheter 12 made from much softer material.

FIG. 3C is a cross-sectional view of the introducer needle 14 and the over-the-needle catheter 12 shown in FIG. 2 with the over-the-needle catheter 12 in a partially advanced position over the distal tip 16 of the introducer needle 14 just as the introducer needle 14 is being removed from the catheter lumen 40 of the over-the-needle catheter 12, shown by the introducer needle moving in the direction of arrow A2. FIG. 3D is a cross-sectional view of the introducer needle 14 and the over-the-needle catheter 12 shown in FIG. 2 after the over-the-needle catheter 12 has been fully advanced into the vasculature of a patient and the introducer needle 14 has been removed from the over-the-needle catheter 12.

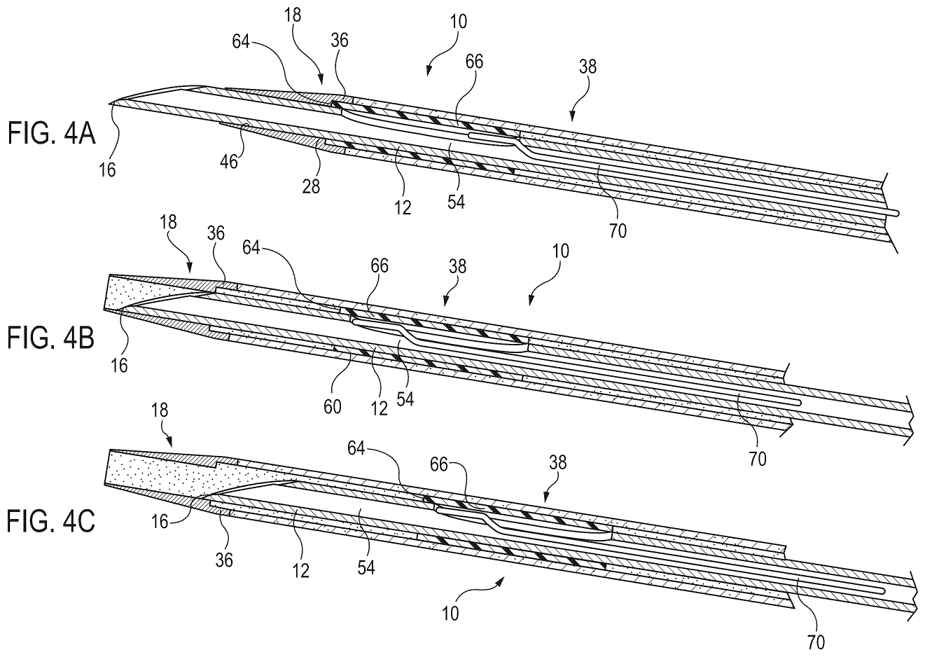

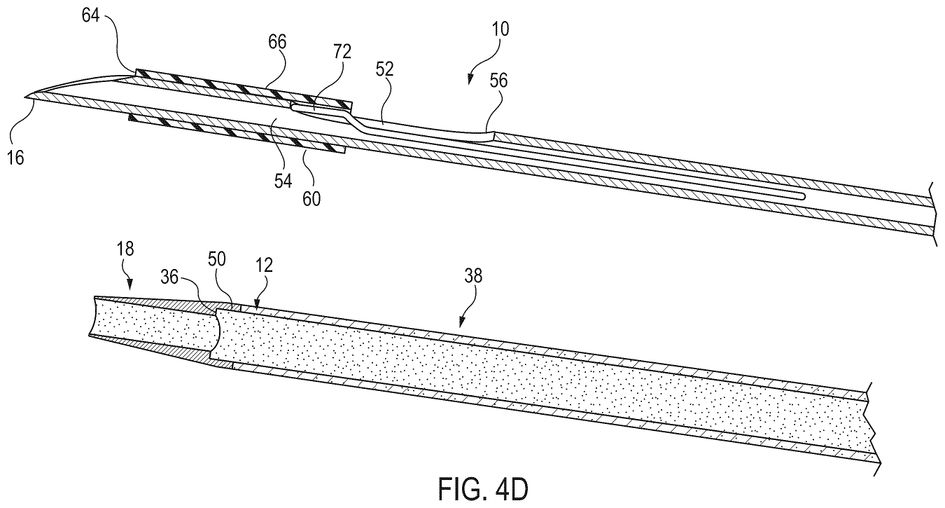

FIGS. 4A-C are alternate views of the catheter assembly 10 shown in FIGS. 2 and 3A-D. FIG. 4A is a cross-sectional view of the introducer needle 14 and the over-the-needle catheter 12 shown in FIG. 2 in a position prior to insertion into a patient. The distal tip 18 of the over-the-needle catheter 12 is in a position so that the distal tip 16 of the introducer needle 14 is exposed to pierce an injection site of a patient. FIG. 4B is a cross-sectional view of the introducer needle 14 and the over-the-needle catheter 12 shown in FIG. 2 with the over-the-needle catheter 12 in a hooded position over the distal tip 16 of the introducer needle 14 as discussed above with respect to FIG. 3B. FIG. 4C is a cross-sectional view of the introducer needle 14 and the over-the-needle catheter 12 shown in FIG. 2 with the over-the-needle catheter 12 in a partially advanced position over the distal tip 16 of the introducer needle 14 and the introducer needle 14 is being withdrawn from the over-the-needle catheter 12.

FIG. 4 D is a cross-sectional view of the introducer needle 14 and the over-the-needle catheter 12 shown in FIG. 2 after the over-the-needle catheter 12 has been placed in the vasculature of a patient and the introducer needle 14 has been removed from the over-the-needle catheter 12.

One method often used during catheter placement is referred to as a “hooding” method. During insertion, upon initial flashback of blood confirmation that the need has punctured the targeted vein, clinicians often pause further needle advancement, and partially advance the catheter far enough for the catheter to cover the needle tip and bevel. This helps prevent the bevel from causing unnecessary vein damage during the remainder of the insertion process. Once the catheter is hooded, the angle is typically reduced or lowered, and the catheter can be fully advanced into the vein. Although hooding is currently used as an insertion method, it is fully based on user skill and technique and is often not effective. According to one or more embodiments, a catheter assembly is provided which establishes a mechanically defined position to allow proper and consistent hooding while providing tip support to be able to successfully advance a catheter with much softer material.

In some of the embodiments shown in FIGS. 2, 3A-D and 4A-C, The catheter assembly 10 further comprises an advancement member 70 configured to advance the catheter distal tip support element 60 and over-the-needle catheter 12. As shown, the advancement member 70 comprises an elongate member extending through the proximal end 17 of the introducer needle 14. In some embodiments, the advancement element 70 has a distal end 72 engaged in a receptacle 66 in the catheter distal tip support element 60. In some embodiments, the advancement member 70 comprises a wire. The advancement member should have sufficient rigidity to advance the over-the-needle catheter 12 as well as some degree of flexibility to complete insertion of the over-the-needle catheter 12 into the vasculature of a patient. A wire having properties similar to a medical guidewire may be suitable according to one or more embodiments.

In FIGS. 4A-D and 5A-B, the catheter assembly 10 utilizes an advancement element that is a guidewire-like member that passes through the needle lumen 54 of the needle to attach to the catheter distal tip support element 60 in the form of a sleeve through the notch 52 of the introducer needle 14. In some embodiments, the notch (or slot) 52 may continue distally to the needle tip opening. The catheter distal tip support element 60 is coupled to the advancement member 70 or may be elongated to also serve as the catheter distal tip support element 60 as described further below.

Thus, it will be appreciated that the catheter distal tip support element 60 comprises a sleeve as shown that slides over the introducer needle 14 and is sandwiched between the sidewall 15 of the needle and the tubular portion 38 of the over-the-needle catheter 12. In some embodiments, the sleeve comprises a sleeve material having a Shore durometer that is equal to or greater than (a harder material) than a Shore durometer than the second material of the tubular portion 38 of the catheter. In some embodiments, the Shore durometer of the sleeve is equal to or greater than the Shore durometer of the distal tip 18 of the over-the-needle catheter 12. The sleeve material can comprise plastic or metal.

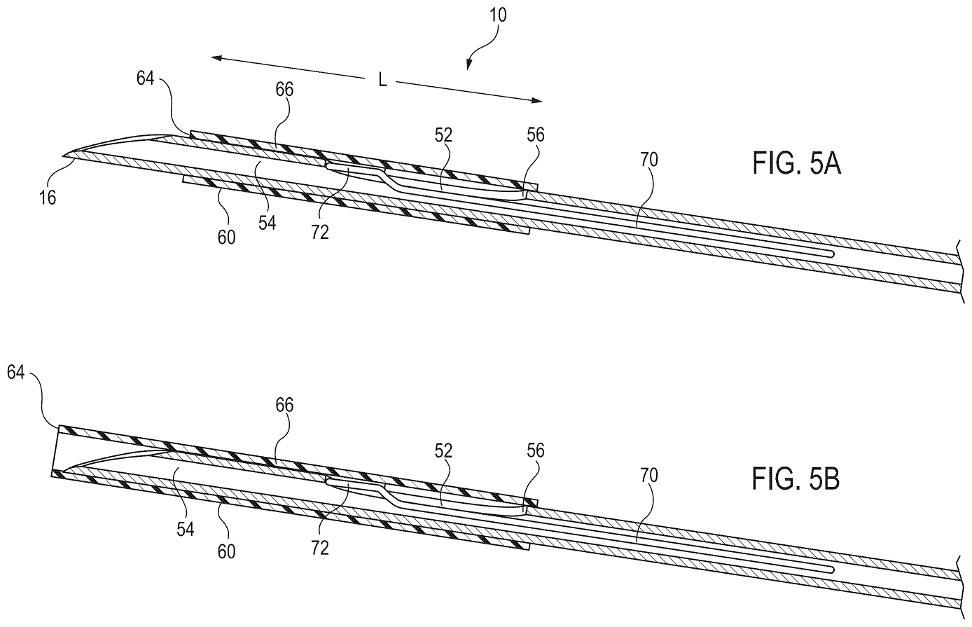

In the embodiment shown in FIGS. 5A and 5B, the sleeve has a length “L” sufficient to cover the notch (or slot) 52 and the distal tip 16 of the introducer needle 14 upon removal of the introducer needle from the catheter. FIG. 5A is a cross-sectional view of a second embodiment of a catheter assembly 10 with the longer reinforced catheter distal tip support element 60 in an initial position. FIG. 5B is a cross-sectional view of the catheter assembly 10 in FIG. 5A showing the longer reinforced catheter distal tip 18 covering the distal tip 16 of the introducer needle 14.

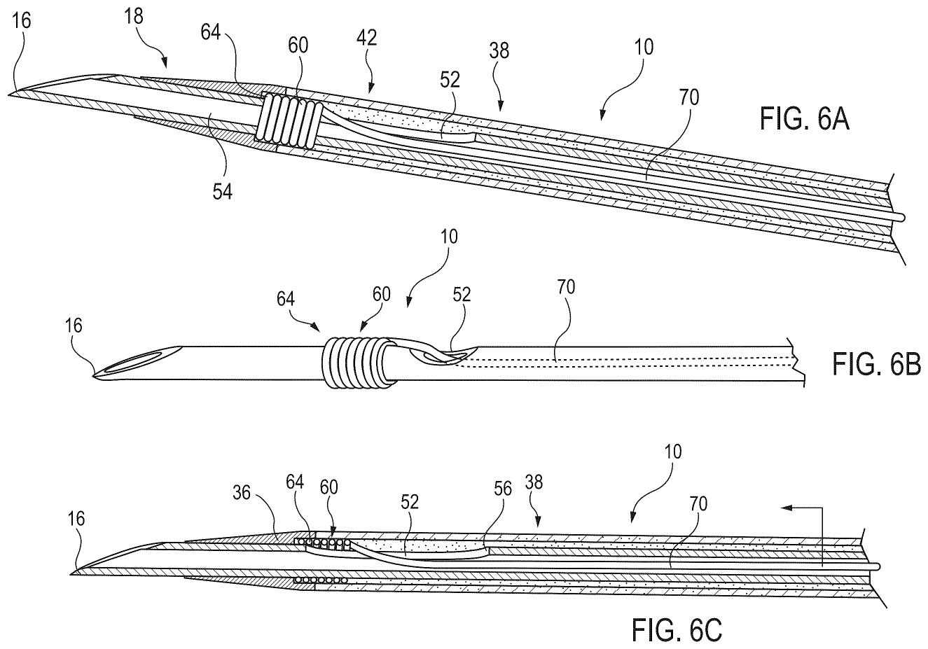

As mentioned above, the catheter distal tip support element 60 is coupled to the advancement member 70 or it may be elongated to also serve as the catheter distal tip support element 60. FIG. 6A shows an embodiment of a catheter assembly

FIG. 6A is a cross-sectional view of a third embodiment of a catheter assembly with a coil element providing the catheter distal tip support element 60. FIG. 6B is a side view of the introducer needle 14 shown catheter assembly in FIG. 6A showing the coil element that provides the catheter distal tip support element 60. the advancement element and the catheter distal support element are integral. In the embodiments, in FIGS. 6A-C, the advancement element comprises a wire, and the wire is fed through the notch (or slot) 52 in the introducer needle 14. The catheter distal tip support element 60 is formed by the coiled wire around the introducer needle 14.

The coil that forms the distal tip support has a distal surface 64 that is configured to engage the shoulder 36 of the distal tip 18 of the over-the-needle catheter 12.

FIG. 6C is a cross-sectional view showing a variant of the catheter assembly shown in FIG. 6A.

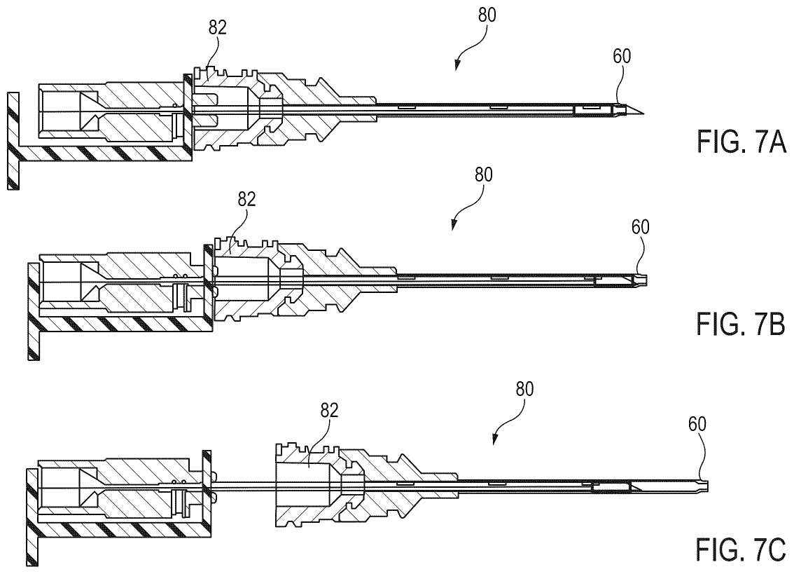

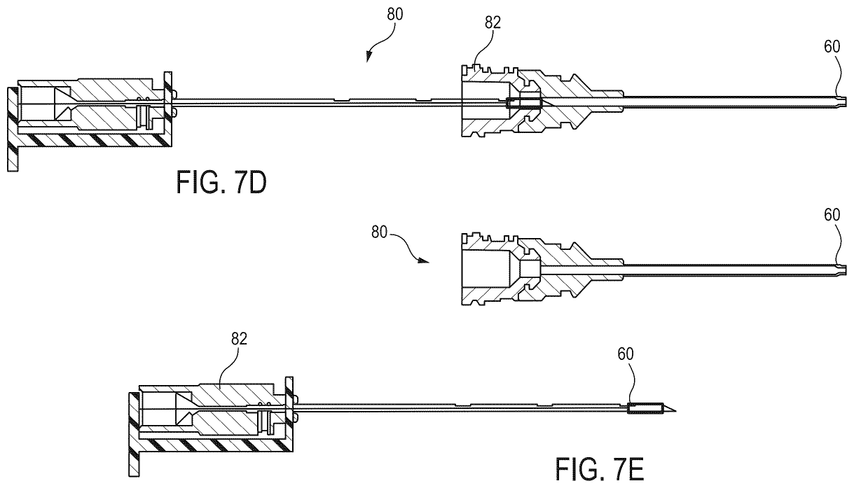

FIGS. 7A-E show a fourth embodiment showing a catheter adapter assembly 80. In FIGS. 7A-E, the displacement force required to hood the distal tip 16 of the introducer needle 14 may be achieved by mechanical displacement between a catheter adapter assembly 80 and needle hub, tip shield, grip, and/or between the tip shield and needle hub among other methods. The catheter distal tip support element 60 may advance with the catheter adapter assembly 80 and may be advanced via the same user applied mechanical separation between the above-mentioned components or by other methods. Other methods may include guidewire type advancement by the user to break tip adhesion and support the catheter while the catheter is being advanced.

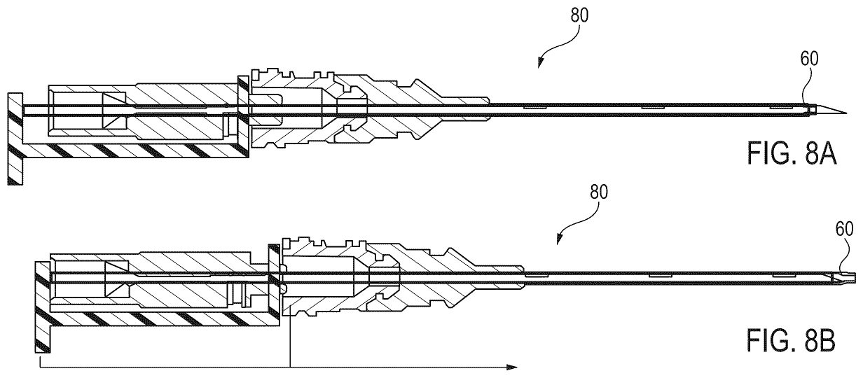

In the case of an elongated distal tip support sleeve without an in-needle advancement member, the elongated sleeve would be advanced by the use during the hooding step when the catheter and catheter adapter are separated from the needle hub, tip shield, or grip as shown in FIGS. 8A-B.

FIG. 7A is a cross-sectional view of a fourth embodiment of a catheter adapter assembly 80 with a catheter distal tip support element 60 in an initial position. FIG. 7B is a cross-sectional view of the catheter adapter assembly 80 in FIG. 7A with the over-the-needle catheter 12 in the hooded position. FIG. 7C is a cross-sectional view of the catheter adapter assembly 80 in FIG. 7A showing the over-the-needle catheter 12 further advanced from the introducer needle 14. FIG. 7D is a cross-sectional view of the catheter adapter assembly 80 in FIG. 7A showing the introducer needle 14 nearly fully withdrawn from the over-the-needle catheter 12 after the over-the-needle catheter 12 has been placed in the vasculature of a patient. FIG. 7E is a cross-sectional view of the catheter adapter assembly 80 in FIG. 7A showing the introducer needle 14 fully withdrawn from the over-the-needle catheter 12 after the over-the-needle catheter 12 has been placed in the vasculature of a patient.

FIG. 8A is a cross-sectional view of a fifth embodiment showing a catheter adapter assembly 80 with a catheter distal tip support in the form of an advancement sleeve instead of an advancement member that passes through the introducer needle 14 to attach to the distal tip support sleeve via a notch 52 in the needle cannula in an initial position. FIG. 8B is a cross-sectional view of the catheter assembly in FIG. 8A with the over-the-needle catheter 12 in the hooded position.

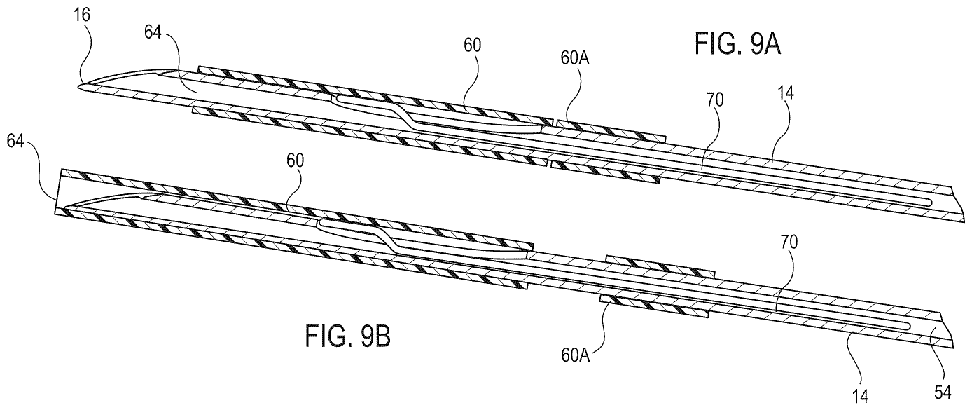

FIG. 9A is cross-sectional view of a sixth embodiment of a catheter assembly 10 with a catheter distal tip support element 60 and a supplemental distal tip support 60A in an initial position. FIG. 9B is a cross-sectional view of the catheter assembly in FIG. 9A with the catheter distal tip support element 60 is shown in the hooded position. In the embodiments in FIGS. 9A and 9B, there may be a case where the catheter distal tip support element 60 may not be stiff enough to hold the insertion forces on the distal tip 18 of the over-the-needle catheter 12, in which case a supplemental distal tip support 60A may be located proximal to the catheter distal tip support element 60. This supplemental distal tip support 60A may be a ferrule that is fixed to the sidewall 15 of the introducer needle 14 proximal to the catheter distal tip support element 60, and in some embodiments, the supplemental distal tip support 60A is not moveable with respect to the introducer needle. In one or more embodiments, the supplemental distal tip support 60A provides additional support to the catheter distal tip support element 60, which is configured to provide an initial insertion force when the catheter assembly is first inserted into a patient.

The catheter distal tip support element 60 is configured transfer the insertion forces to the supplemental distal tip support 60A which is fixed to the sidewall 15 of the introducer needle 14, thus unloading the supplemental distal tip support 60A. The supplemental distal tip support 60A can still advance the catheter distal tip support element 60 for hooding the distal tip 16 of the needle as shown below in FIG. 9B. The supplemental distal tip support 60A can be fixed to the sidewall 15 of the introducer needle 14 by laser welding, adhesive or any other means. The supplemental distal tip support 60A may be provided with a crimp in the sidewall 15 that prevents the distal movement of the catheter distal tip support element 60. The supplemental distal tip support 60A may also be provided by a step on the outside wall cannula or any other means of enlarging the outside of the cannula. The supplemental distal tip support 60A may also be a second, blunt cannula that covers the proximal end of the introducer needle 14.

The catheter assemblies described in this disclosure may be applied to a wide variety of medical devices. They may be applied to peripheral intravenous catheters (PIVC), long PIVC, mini-midline, midline, arterial, and port access vascular access devices. Embodiments of the catheter assemblies may be used with a variety of needle safety features, including a v-clip, simple clip, spring retraction, saf-t-intima type safety, among others. Hooding advancement according to one or more embodiments may occur via user input (distal rotational, proximal rotational, squeeze, button, slider, push button with spring element).

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A catheter assembly configured for insertion into vasculature of a patient, the catheter assembly comprising:
a catheter, comprising:
a catheter stepped surface having a distal surface with a first inner diameter, a proximal surface with a second inner diameter greater than the first inner diameter, and a shoulder disposed between the distal surface and the proximal surface; and
an elongate tubular portion proximate the catheter stepped surface and having the second inner diameter of the proximal surface, wherein the shoulder, the proximal surface, and the distal surface are constructed of a first material having a Shore durometer equal to or greater than a Shore durometer of a second material of the elongate tubular portion, the catheter having a catheter lumen extending therethrough, wherein the catheter stepped surface is disposed within the catheter lumen; and
an introducer needle disposed within the catheter lumen, the introducer needle comprising a sidewall defining a proximal end, a distal tip, and a lumen extending through from the proximal end to the distal tip; and
a catheter distal tip support element movably disposed within the catheter lumen and over the introducer needle, the catheter distal tip support element comprising a support material having a higher Shore durometer than the second material of the elongate tubular portion of the catheter, the catheter distal tip support element movable with respect to the catheter and the introducer needle and having a support distal surface configured to contact the shoulder of the catheter and to advance the catheter in distal direction and prevent kinking or buckling of the second material of the elongate tubular portion as the catheter is advanced into the vasculature of the patient.

2. The catheter assembly according to claim 1, further comprising an advancement member configured to advance the catheter distal tip support element and the catheter.

3. The catheter assembly according to claim 2, wherein the advancement member comprises an elongate member extending through the proximal end of the introducer needle.

4. The catheter assembly according to claim 3, wherein the advancement member is engaged in a receptacle in the catheter distal tip support element.

5. The catheter assembly according to claim 4, wherein the advancement member comprises a wire.

6. The catheter assembly of claim 5, wherein the introducer needle comprises a notch in the sidewall, and the wire extends through the notch.

7. The catheter assembly according to claim 3, wherein the advancement member and the catheter distal tip support element are integral.

8. The catheter assembly according to claim 7 wherein the advancement member comprises a wire.

9. The catheter assembly according to claim 7, wherein the catheter distal support element comprises a coil surrounding the introducer needle.

10. The catheter assembly according to claim 4, wherein the catheter distal tip support element comprises a sleeve.

11. The catheter assembly according to claim 10, wherein the sleeve comprises a sleeve material having a Shore durometer that is greater than the Shore durometer than the second material.

12. The catheter assembly according to claim 11, wherein the sleeve material comprises plastic.

13. The catheter assembly according to claim 11, wherein the sleeve material comprises metal.

14. The catheter assembly according to claim 10, wherein the sleeve has a length sufficient to cover a notch in the introducer needle and the distal tip of the introducer needle upon removal of the introducer needle from the catheter.

15. The catheter assembly according to claim 1, wherein the catheter distal support element comprises an elongate sleeve disposed over the introducer needle having a length that extends to the proximal end of the introducer needle.

16. The catheter assembly according to claim 1, further comprising supplemental distal tip support located proximal to the catheter distal tip support element.

17. The catheter assembly according to claim 16, wherein the supplemental distal tip support is fixed to the sidewall of the introducer needle.

18. The catheter assembly according to claim 17, wherein the supplemental distal tip support is not moveable with respect to the introducer needle.

19. The catheter assembly according to claim 18, wherein the supplemental distal tip support is configured to provide additional support to the catheter distal tip support element.

20. The catheter assembly according to claim 19, wherein the supplemental distal tip support comprises a ferrule.
